(19) United States
(12) Patent Application Publication
(10) Pub. No.: US 2010/0166870 A1
(43) Pub. Date: Jul. 1, 2010

(54) Method for Coating Nanoparticles

(75) Inventors: Swaminathan Iyer, Crawley (AU); Colin Raston, South Yunderup (AU)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: THE UNIVERSITY OF WESTERN AUSTRALIA, Nedlands (AU)

(21) Appl. No.: 12/520,498

(22) PCT Filed: Dec. 20, 2007
(86) PCT No.: PCT/AU2007/001976

§ 371 Date: Jan. 6, 2010

(30) Foreign Application Priority Data
Dec. 21, 2006 (AU) 2006907181

(51) Int. Cl. A61K 9/14; B05D 7/00

(52) U.S. Cl. 424/490; 427/212; 427/216; 427/215; 977/742; 977/773; 977/847; 977/890; 977/906

(57) ABSTRACT

A method of coating nanoparticles comprising subjecting nanoparticles, a coating precursor and one or more reagents to shear, wherein the coating precursor and the one or more reagents react to provide a coating on the nanoparticles.

### FIELD OF THE INVENTION

The present invention relates to a method of coating nanoparticles including nanotubes and nanoparticles of therapeutic compositions. The invention also relates to coated nanoparticles.

### BACKGROUND ART

Carbon nanotubes (CNTs) have been the subject of numerous scientific investigations over the last decade because of their exceptional properties. But it is their inherent nano-dimensionality that makes their application problematic. Issues pertaining to the problems of processability and scalability, in order to develop their true commercial and technological potential, arise from their tendency to aggregate during processing, the difficulty in controlling their length, imparting “wettability” in order to coat them with other materials, and the high cost of their production.

Carbon nanotubes interacting with metals are prime candidates for a wide gamut of applications such as sensing materials, catalysts, Field-Effect Transistors (FET) and fabrication of novel nanoelectromechanical systems (NEMS). Thus far most of the reports for coating CNTs have involved high temperature/high pressure techniques (e.g. electron beam evaporation coating), and attempts to manipulate the longitudinal dimensions of CNTs involving the use of toxic chemicals. Controlling the size of metal particles attached to CNTs and the continuity of metal coatings on CNTs, along with their structural integrity, is a major challenge in gaining access to new metal-CNT hybrids. Indeed, the axiom “quasi-continuous coating of metal” has become associated with most of the publications on metal-CNT hybrids.

A recent systematic Density Functional Theory study and experimental work on the interplay between CNTs and various metal atoms and clusters (Ti, Ni, Pt, Pd, Au) revealed that while metals like Pd and Ti formed quasi-continuous coatings, Au forms only discrete particles on the surface of CNTs. Factors that limit the wettability of CNTs by metals are the low chemical reactivity, surface curvature, small diameter and large aspect ratio of the CNTs, and accordingly it is regarded as very difficult to make a continuous plating layer of metals on CNTs.

Poor bioavailability is a significant problem encountered in the development of therapeutic compositions, particularly those compounds containing an active agent that is poorly soluble in water. An active agent's bioavailability is the degree to which the active agent becomes available to the target tissue in the body after systemic administration through, for example, oral or intravenous means. Many factors may affect bioavailability, including the form of dosage and the solubility and dissolution rate of the active agent.

Reducing the size of particulates of drugs to the nano-meter can dramatically improve dissolution velocity and greatly enhance their in vivo bioavailability and in vitro dissolution velocity. This is important for drugs that are otherwise poorly soluble in water

Increased bioavailability can circumvent the need for many drugs to be taken with food. Furthermore, administering drugs in nanoparticulate form (high efficacy/minimum intake) may overcome the common side effects (ranging from fatigue/weakness to gastrointestinal symptoms) associated with drugs in their traditional form (low efficacy/maximum intake). Formulating the drug as nanoparticles is also beneficial to the end user, and is also beneficial as it provides prolonged patent protection from generic manufacturers. Fabricating nanoparticles can apply to either new drug candidates or to existing marketed products for improving their performance and value.

The preceding discussion of the background to the invention is intended to facilitate an understanding of the present invention. However, it should be appreciated that the discussion is not an acknowledgement or admission that any of the material referred to was part of the common general knowledge in Australia as at the priority date of the application.

Throughout the specification, unless the context requires otherwise, the word “comprise” or variations such as “comprises” or “comprising”, will be understood to imply the inclusion of a stated integer or group of integers but not the exclusion of any other integer or group of integers.

### DISCLOSURE OF THE INVENTION

Those skilled in the art will appreciate that the invention described herein is susceptible to variations and modifications other than those specifically described.

It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference.

“Therapeutically effective amount” as used herein with respect to a drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that drug dosages are, in particular instances, measured as oral dosages, or with reference to drug levels as measured in blood.

In its broadest aspect, the invention provides a method of coating nanoparticles. The invention also provides coated nanotubes and a method of coating nanotubes. Nanoparticles of metal-metal, metal-metal compound, and metal compound-metal compound composite materials, and methods for preparing said nanoparticles are provided. Furthermore, coated pharmaceutical nanoparticles are also provided.

Accordingly, in a first aspect, the present invention provides a method of coating nanoparticles comprising subjecting nanoparticles, a coating precursor, and one or more reagents capable of reacting with the coating precursor and preparing the coating, to shear, thereby causing production of coated nanoparticles from said reaction under shear.

In one embodiment of the invention, the nanoparticles, the coating precursor, and the one or more reagents are subjected to shear on a rotating surface of a rotating surface reactor. Typically, the nanoparticles, the coating precursor, and the one or more reagents are directed separately to the rotating surface of the rotating surface reactor, although it will be appreciated that the one or more reagents, or the coating precursor, can be combined with the nanoparticles prior to subjecting them to shear.

The rotating surface reactor is operated so that the rotating surface spins at a speed sufficient to cause the combined liquid dispersion of nanoparticles, coating precursor-containing solution, and the one or more reagents to spread over the rotating surface as a continuously flowing thin film.

In a preferred embodiment, the nanoparticles are provided to the rotating surface reactor as a liquid dispersion thereof.

In one embodiment the nanoparticles are nanotubes.

Accordingly, in a further aspect, the invention provides coated nanotubes.

In one embodiment the coated nanotubes are substantially continuously coated.

An inherent advantage of the process for coating nanotubes is that the coated nanotubes are cut into shorter lengths as the reaction proceeds. Thus, in a further aspect the invention provides a process for cutting nanotubes comprising preparing coated nanotubes and subjecting said coated nanotubes to shear.

In one embodiment of the invention the coated nanotubes are metal coated nanotubes. The coated nanotubes are prepared in accordance with the process of the first aspect of the invention.

In a further aspect, the invention provides a method of preparing nanoparticles of a first metal and a second metal comprising subjecting nanoparticles of a first metal, a second metal precursor and one or more reagents capable of reacting with the coating precursor and preparing the coating, to shear, thereby causing production of nanoparticles of the first and second metal compounds from said reaction under shear.

In one embodiment the first metal is silver. In another embodiment the second metal is gold.

In a further aspect, the invention provides nanoparticles comprising a core of silver and gold coating.

In a further aspect, the invention provides metal-metal compound nanoparticles.

In a further aspect, the invention provides a method for preparing metal-metal compound nanoparticles comprising subjecting metal nanoparticles, a metal compound precursor; and one or more reagents capable of reacting with the metal compound precursor and preparing the metal compound, to shear, thereby causing production of metal-metal compound nanoparticles from said reaction under shear.

In a further aspect, the invention provides nanoparticles of a first metal compound and a second metal compound.

In a further aspect, the invention provides a method for preparing nanoparticles of a first metal compound and a second metal compound comprising subjecting nanoparticles of a first metal compound, a second metal compound precursor, and one or more reagents capable of reacting with the second metal compound precursor and preparing the second metal compound, to shear, thereby causing production of nanoparticles of the first and second metal compounds from said reaction under shear.

In a further aspect, the invention provides dendritic nanoparticles.

In an further aspect, the invention provides a process for preparing dendritic nanoparticles comprising subjecting nanotubes, a coating precursor, the concentration of the coating precursor being sufficient to promote growth in respective lateral and axial directions from activation sites on the nanotubes, and one or more reagents capable of reacting with the coating precursor and preparing the coating, to shear, thereby causing production of a dendritic coating on the nanotubes from said reaction under shear.

In a further aspect, the invention provides coated therapeutic nanoparticles.

In a further aspect, the invention relates to the use of a coated therapeutic composition in the manufacture of a medicament.

In a further aspect, the invention relates to methods of treatment of an animal comprising administering to said animal a therapeutically effective amount of a coated therapeutic composition produced according to a method of the invention, wherein said animal is in need of said therapeutically active agent.

In a further aspect, the invention relates to the product of the aforementioned methods and its use in the preparation of medicaments and therapeutically active compositions suitable for treating an animal, such as a human. The invention includes methods for preparing medicaments and pharmaceutically acceptable compositions comprising the purified nano-particulate therapeutically active agent.

### BRIEF DESCRIPTION OF THE DRAWINGS

FIG. 1 is a schematic diagram showing the process steps relating to the preparation of coated nanoparticles, illustratively in respect to the preparation of metal-coated carbon nanotubes, in accordance with the present invention;

FIG. 2 is a series of high resolution transmission electron microscopy (TEM) micrographs of metal-coated carbon nanotubes prepared in accordance with the process of the present invention with various metal precursor concentrations (a) Au (1 mM), (b) Pt (0.5 mM), (c) Au (10 mM), and (d) Ag (10 mM);

FIG. 3 is a series of TEM micrographs of gold-coated carbon nanotubes prepared in accordance with the process of the present invention;

FIGS. 4(a) and 4(b) are TEM micrographs of dendritic silver-coated carbon nanotubes;

FIGS. 5(a)-(c) are a series of TEM micrographs of gold-coated carbon nanotubes;

FIGS. 6(a), 6(b) and 6(g) are a series of TEM micrographs of silver-coated one dimensional nanoparticles of fullerene C₆₀;

FIGS. 6(c)-(f), and 6(h) are a series of FFT patterns of the silver-coated one dimensional nanoparticles of fullerene C₆₀ of FIG. 4;

FIG. 7 is a series of TEM micrographs of gold-coated silver nanoparticles;

FIG. 8 is a TEM micrograph of a titania-coated silver nanoparticle;

FIGS. 9(a) and 9(b) are TEM micrographs of a hollow titania nanoparticle and a titania-coated iron oxide nanoparticle, respectively; and

FIG. 9(c) shows several UV-visible absorption spectra for liquid dispersions of silver nanoparticles, titania-coated silver nanoparticles, hollow titania nanoparticles, and titania-coated iron oxide nanoparticle, respectively.

### BEST MODE(S) FOR CARRYING OUT THE INVENTION

In one embodiment of the invention, coated nanoparticles prepared in accordance with the methods of the present invention comprise a core and a coating. Typically the core has a mean particle size in a range of 5-100 nm or a cross-sectional diameter in a range of 5-100 nm, and the thickness of the coating may vary in a range of 5-100 nm.

The coated nanoparticles prepared according to the methods of the present invention exhibit a narrow particle size distribution and also show a high degree of uniformity in shape, Particularly if the preformed nanoparticles exhibit a narrow size distribution. It will be appreciated that in some embodiments of the invention, the shape of the coated nanoparticles will be determined by the shape of the core. For example, spheroidal coated nanoparticles can be prepared by coating spheroidal nanoparticles in accordance with the methods of the present invention, and one dimensional coated nanoparticles can be similarly prepared by coating one dimensional nanoparticles or nanotubes. However, in alternative embodiments of the invention, the shape of the coating can be varied irrespective of the shape of the core. For example, the coating on a nanotube can be varied from a coating of discrete nanoparticles, continuous coatings, and dendritic nanostructures.

In one embodiment of the invention, the core of the first material is selected from metals, alloys, metalloids, metal compounds such as metal oxides, inorganic compounds, and carbon-based materials, in particular carbon nanotubes, one-dimensional nanoparticles of fullerene C₆₀, and three-dimensional nanoparticles of fullerene C₇₀ and therapeutic compositions.

Suitable examples of metals which can comprise the core of the coated nanoparticle include, but are not limited to, noble or a platinum metal such as Ag, Au, Pd, Pt, Rh, Ir, Ru, and Os, transition metals such as Ti, Cr, Mn, Fe, Co, Ni, Cu, Zn, Zr, Nb, Mo, Ta, W, Re, and main group metals such as Al, Ga, In, Si, Ge, Sn, Sb, Bi, Te. It will be appreciated that some main group metals, in particular Si and Ge, are also commonly referred to as metalloids.

Suitable examples of alloys which can comprise the core of the coated nanoparticle include, but are not limited to, alloys of noble or platinum metal and transition metals, in particular alloys of silver and transition metals such as Ag/Ni, Ag/Cu, Ag/Co, and platinum and transition metals such as Pt/Cu, or noble or platinum alloys such as Ru/Pt.

Non-limiting examples of inorganic compounds which can comprise the core of the coated nanoparticle include, but are not limited, to SiO₂, metal compounds, in particular metal oxides such as TiO₂ and iron oxides.

Suitable examples of therapeutic compositions which can comprise the core of the coated nanoparticles include, but are not limited to, biologics, amino acids, proteins, peptides, nucleotides, nucleic acids, and analogs thereof. Further, the therapeutic composition may be selected from a variety of classes of drugs, including anti-obesity drugs, central nervous system stimulants, carotenoids, corticosteroids, elastase inhibitors, anti-fungals, oncology therapies, anti-emetics, analgesics, cardiovascular agents, anti-inflammatory agents, such as NSAIDs and COX-2 inhibitors, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, alpha-adrenergic receptor blocking agents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines. In other aspects, the precursor compound may be selected from haloperidol, DL isoproterenol hydrochloride, terfenadine, propranolol hydrochloride, desipramine hydrochloride, salmeterol, sildenafil citrate, tadalafil, vardenafil, fenamic acids, Piroxicam, Naproxen, Voltaren (diclofenac), rofecoxib, ibuprofren ondanstetron, sumatriptan, naratryptan, ergotamine tartrate plus caffeine, methylsegide, olanzapine.

A description of classes of active agents and a listing of species within each class can be found in Martindale's The Extra Pharmacopoeia, 31st Edition (The Pharmaceutical Press, London, 1996), specifically incorporated by reference. Another source of active agents is the Physicians Desk Reference (60^{th} Ed., pub. 2005), familiar to those of skill in the art. The active agents are commercially available and/or can be prepared by techniques known in the art.

An exhaustive list of drugs for which the methods of the invention are suitable would be burdensomely long for this specification. However, reference to the general pharmacopoeia listed above would allow one of skill in the art to select virtually any drug to which the method of the invention may be applied. Notwithstanding the general applicability of the method of the invention, more specific examples of therapeutic compositions include, but are not limited to: haloperidol (dopamine antagonist), DL isoproterenol hydrochloride (β-adrenergic agonist), terfenadine (H1-antagonist), propranolol hydrochloride (β-adrenergic antagonist), desipramine hydrochloride (antidepressant), salmeterol (b2-selective adrenergic agonist), sildenafil citrate, tadalafil and vardenafil. Minor analgesics (cyclooxygenase inhibitors), fenamic acids, Piroxicam, Cox-2 inhibitors, and Naproxen, and others, may all benefit from being prepared in a nanoparticle composition. In addition, some active agents may have the benefit of absorption through the skin if presented in a nanoparticle formulation. Such drugs include, but are not limited to, Voltaren (diclofenac), rofecoxib, and Ibuprofen.

Other therapeutic compositions may be used to prepare nanoparticulate microstructures for treating migraines and other psychotropic disorders, including, for example, 5-hydroxytryptamine receptor antagonists (e.g. ondanstetron, sumatriptan, naratryptan), ergotamine tartrate plus caffeine, or methylsegide.

Further suitable examples of therapeutic compositions which can comprise the core of the coated nanoparticle include, but are not limited to, neproxeb, fenofribate and megestrol as shown below:

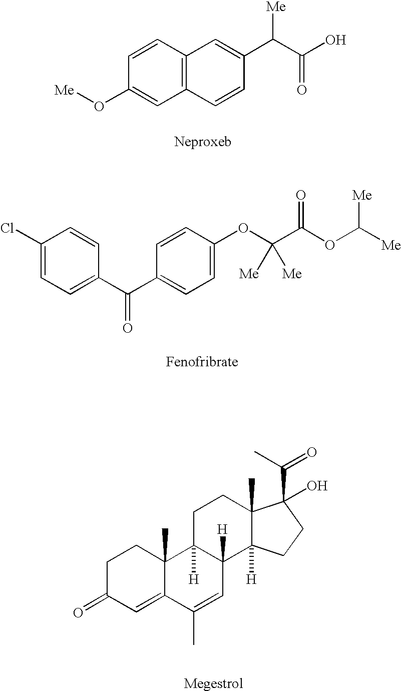

Further suitable examples of therapeutic compositions which can comprise the core of the coated nanoparticle also include, but are not limited to, siRNA, siDNA, nucleotides, and peptides.

In one embodiment of the invention, the coating of the second material is selected from a group comprising metals, alloys, metalloids, metal oxides, inorganic compounds and organic compounds.

Suitable examples of metals which can comprise the coating of the coated nanoparticle include, but are not limited to, noble or a platinum metal such as Ag, Au, Pd, Pt, Rh, Ir, Ru, and Os, transition metals such as Ti, Cr, Mn, Fe, Co, Ni, Cu, Zn, Zr, Nb, Mo, Ta, W, Re, and main group metals such as Al, Ga, In, Si, Ge, Sn, Sb, Bi, Te. It will be appreciated that some main group metals, in particular Si and Ge, are also commonly referred to as metalloids.

Suitable examples of alloys which can comprise the coating of the coated nanoparticle include, but are not limited to, alloys of noble or platinum metal and transition metals, in particular alloys of silver and transition metals such as Ag/Ni, Ag/Cu, Ag/Co, and platinum and transition metals such as Pt/Cu, or noble or platinum alloys such as Ru/Pt.

Non-limiting examples of inorganic compounds which can comprise the coating of the coated nanoparticle include, but are not limited, to SiO₂, metal compounds, in particular metal oxides such as TiO₂ and iron oxides such as magnetite and maghemite.

Where the nanoparticle is a therapeutic composition, the coating may comprise a polymer. Suitable examples of polymers which can comprise the coating of the coated nanoparticle include, but are not limited to, hydroxypropyl methylcellulose, hydroxypropyl cellulose, methylcellulose, ethyl cellulose pseudolatex; and (iii) Enteric polymer coatings—cellulose acetate phthalate (Aquacoat®), polyvinyl acetate phthalate (Sureteric®), hydroxypropyl methylcellulose acetate succinate (AQOAT®), and Eudragit®.

The present invention also relates to the use of the composition of the invention in the manufacture of a medicament. Such a medicament may include the composition alone or more preferably the composition may be combined with one or more pharmaceutically acceptable carriers, as well as any desired excipients or other like agents commonly used in the preparation of pharmaceutically acceptable compositions.

As used herein “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral administration, intravenous, intraperitoneal, intramuscular, sublingual, transdermal or oral administration. Pharmaceutically acceptable carriers include sterile aqueous solutions or dispersions and sterile powders for the extemporaneous preparation of sterile injectable solutions or dispersion. The use of such media and agents for the manufacture of pharmaceutical compositions is well known in the art. Except insofar as any conventional media or agent is incompatible with the nano-particular preparation, use thereof in the manufacture of a pharmaceutical composition according to the invention is contemplated.

Where the nanoparticle is a therapeutic composition, the coated nanoparticle may further comprise excipients for stabilization of nano-particles. Suitable examples of excipients include, but are not limited to, Tween 80, poloxamer, and lecithin.

It will be appreciated that the nanoparticle core may comprise more than one therapeutic composition.

Where the nanoparticle is a therapeutic composition, the coated nanoparticle may further comprise one or more of the following examples:
(1) polymeric surface stabilizers including, but are not limited to polyethylene glycol (PEG), polyvinylpyrrolidone (PVP), polyvinylalcohol, corspovidone, polyvinylpyrrolidone-polyvinylacytate copolymer, cellulose derivatives, hydroxypropylmethyl cellulose, hydroxypropyl cellulose, carboxymethylethyl cellulose, hydroxypropyllmethyl cellulose phthalate, polyacrylates and polymethacrylates, urea, sugars, polyols, and their polymers, emulsifiers, sugar gum, starch, organic acids and their salts, vinyl pyrrolidone and vinyl acetate; and/or
(2) binding agents such as various celluloses and cross-linked polyvinylpyrrolidone, microcrystalline cellulose; and/or
(3) filling agents such as lactose monohydrate, lactose anhydrous, and various starches; and/or
(4) lubricating agents such as agents that act on the flowability of the powder to be compressed, including colloidal silicon dioxide, talc, stearic acid, magnesium stearate, calcium stearate, silica gel; and/or
(5) sweeteners such as any natural or artificial sweetener including sucrose, xylitol, sodium saccharin, cyclamate, aspartame, and accsulfame K; and/or
(6) flavouring agents; and/or
(7) preservatives such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride; and/or
(8) buffers; and/or
(9) Diluents such as pharmaceutically acceptable inert fillers, such as microcrystalline cellulose, lactose, dibasic calcium phosphate, saccharides, and/or mixtures of any of the foregoing; and/or
(10) wetting agents such as corn starch, potato starch, maize starch, and modified starches, croscarmellose sodium, crosspovidone, sodium starch glycolate, and mixtures thereof; and/or
(11) disintegrants; and/or
(12) effervescent agents such as effervescent couples such as an organic acid (e.g., citric, tartaric, malic, fumaric, adipic, succinic, and alginic acids and anhydrides and acid salts), or a carbonate (e.g. sodium carbonate, potassium carbonate, magnesium carbonate, sodium glycine carbonate, L-lysine carbonate, and arginine carbonate) or bicarbonate (e.g. sodium bicarbonate or potassium bicarbonate); and/or
(13) other pharmaceutically acceptable excipients.

Therapeutic compositions suitable for use in animals and in particular in man typically must be sterile and stable under the conditions of manufacture and storage. The pharmaceutical composition comprising nanoparticles can be formulated as a solution, microemulsion, liposome, or other ordered structure suitable to high drug concentration. Actual dosage levels of the therapeutically active agent in the therapeutic composition of the invention may be varied in accordance with the nature of the therapeutically active agent, as well as the potential increased efficacy due to the advantages of providing and administering the active agent in nanoparticle form (e.g., increased solubility, more rapid dissolution, increased surface area of the drug in nanoparticle form, etc.). Thus as used herein “therapeutically effective amount” will refer to an amount of nanocomposite microstructure composition required to effect a therapeutic response in an animal. Amounts effective for such a use will depend on: the desired therapeutic effect; the route of administration; the potency of the therapeutically active agent; the desired duration of treatment; the stage and severity of the disease being treated; the weight and general state of health of the patient; and the judgment of the prescribing physician.

Where the nanoparticle is a therapeutic composition, the coating may comprise further therapeutic composition. The further therapeutic composition may be the same therapeutic composition or a different therapeutic composition including a salt or neutral analogue of the core therapeutic compound.

In one embodiment of the invention, the coated nanoparticles comprise a carbon-based material core and a metal coating. Suitable examples of said coated nanoparticles include, but are not limited to, gold-coated, silver-coated, and platinum-coated carbon nanotubes, silver-coated one dimensional nanoparticles of fullerene C₆₀, and silver-coated three-dimensional nanoparticles of fullerene C₇₀. Interestingly, for the three-dimensional nanoparticles of fullerene C₇₀, the silver coating is disposed on an inner surface thereof.

In a further embodiment of the invention, the coated nanoparticles comprise a core of a first metal and a coating of a second metal. Suitable examples of said coated nanoparticles include, but are not limited to, gold-coated silver nanoparticles.

In a further embodiment of the invention, the coated nanoparticles comprise a metal core and a metal compound coating. Suitable examples of said coated nanoparticles include, but are not limited to, titania-coated silver nanoparticles.

In a still further embodiment of the invention, the coated nanoparticles comprise a core of a first metal compound and a coating of a second metal compound. Suitable examples of said coated nanoparticles include, but are not limited to, titanic-coated iron oxide nanoparticles.

In accordance with the methods of the present invention, nanoparticles, a coating precursor and one or more reagents capable of reacting with the coating precursor and preparing the coating are subjected to shear.

The nanoparticles are typically provided as a liquid dispersion thereof. It will be appreciated that the nanoparticles may be provided in combination with an anti-agglomeration agent to ensure that the nanoparticles are substantially mono-dispersed and to prevent a substantial agglomeration of the nanoparticles. The anti-agglomeration agent binds to the surface of the nanoparticle through strong bonds, or weak secondary bonds, albeit the bonds being sufficiently strong to substantially withstand removal by a solvent in which the anti-agglomeration agent is soluble. The anti-agglomeration agent may be present as a continuous coating on the surface of the nanoparticle or on only a portion of the surface of the nanoparticle.

In some embodiments anti-agglomeration agents are selected from a group comprising tartaric acid, glutathione, arginic acid, biopolymers, cyclodextrins and polysubstituted cyclodextrins, cavitands and polysubstituted cavitands, long chain surfactants and long chain polysubstituted surfactants. Suitable examples of biopolymers include, but are not limited to, starch, modified starch, chitosan, modified chitosan, polysaccharides and derivatives thereof, and gelatin. Suitable examples of cyclodextrins include, but are not limited to, α-cyclodextrin, β-cyclodextrin, γ-cyclodextrin. Suitable examples of cavitands and polysubstituted cavitands include, but are not limited to, calix[n]arenes (n=4, 5, 6, 7, 8), sulphonato-calix[n]arenes (n=4, 5, 6, 7, 8), phosphonato-calix[n]arenes (n=4, 5, 6, 7, 8), O-alkylatedsulphonato-calix[n]arenes (n=4, 5, 6, 7, 8), and calixresorcinarenes and derivates thereof. Suitable examples of long chain polysubstituted surfactants include, but are not limited to, long chain polyhydroxylated surfactants (e.g. PEG), polyphenol, polysubstituted phenols, polyvinylpyrollidone, and polyvinylpyridine.

In alternative embodiments, the anti-agglomeration agents are complexes of iodine and a saccharide, in particular complexes of iodine and starch. Other suitable examples of saccharides include, but are not limited to, chemically modified analogues of starch, oligosaccharides such as cyclodextrins and polysubstituted cyclodextrins, chitin and chemically modified analogues thereof, and chitosan and chemically modified analogues thereof, all capable of forming complexes with iodine.

The nanoparticles are selected according to which type of core is desired for the coated nanoparticle, either with regard to shape or material.

For instance, coated nanotubes can be prepared by subjecting nanotubes, a coating precursor-containing solution and one or more reagents capable of reacting with the coating precursor and preparing the coating, to high shear.

Nanotubes are typically small cylinders made of organic or inorganic materials. Known types of nanotubes include carbon nanotubes, inorganic nanotubes and peptidyl nanotubes. Inorganic nanotubes include WS₂ and metal oxide nanotubes such as oxides of titanium and molybdenum. Preferably the nanotubes are carbon nanotubes (CNTs).

CNTs are sheets of graphite that have been rolled up into cylindrical tubes. The basic repeating unit of the graphite sheet consists of hexagonal rings of carbon atoms, with a carbon-carbon bond length of about 1.45 Å. Depending on how they are made, the nanotubes may be single-walled nanotubes (SWNTs), double walled carbon nanotubes (DWNTs) and/or multi-walled nanotubes (MWNTs). A typical SWNT has a diameter of about 0.7 to 1.4 nm.

The structural characteristics of nanotubes provide them with unique physical properties. Nanotubes may have up to 100 times the mechanical strength of steel and can be up to several mm in length. They exhibit the electrical characteristics of either metals or semiconductors, depending on the degree of chirality or twist of the nanotube. Different forms of nanotubes are known as armchair, zigzag and chiral nanotubes. The electronic properties of carbon nanotubes are determined in part by the diameter and therefore the ‘form’ of the nanotube.

The spaces between the graphite layers of carbon nanotubes, local disorders arising from their high defective structures and the central core should permit large insertion capacity. Due to their characteristics of high stability, low mass density, low resistance, high accessible surface area and narrow pore size distribution, carbon nanotubes are suitable materials for electrochemical capacitors.

It is believed that the uses and applications for carbon nanotubes would be further enhanced by providing coatings of metal thereon. The widely acknowledged difficulties associated with preparing metal coated CNTs have been previously discussed.

The present invention, however, provides a method for the preparation of coated CNTs including metal coated CNTs. While not wishing to be bound by theory, the inventors opine that preparation of the metal coated CNTs in a region of shear overcomes the aforesaid difficulties because of enhanced mixing and mass/heat transfer rates generated in the region of shear. It will be appreciated that CNTs can be continuously coated with metal by employing the methods of the present invention.

A chemical synthetic method for coating SWCNTs with metal (e.g. Ag, Au, Pt) herein is broadly based on the reduction of positive valency metal species (e.g. AgNO₃, HAuCl₄, and HPtCl₆) with a reductant (e.g. ascorbic acid) in the presence of an aqueous dispersion of SWCNTs and an anti-agglomeration agent (e.g. iodine-starch complex), under shear conditions.

Transmission electron micrographs (TEM) confirmed that manipulating the coating of metals (Pt, Au and Ag) on SWCNTs was efficiently attained by regulating the concentration of the positive valency metal species in the feed to the region of high shear. While lower concentrations of the positive valency metal species resulted in the decoration of SWCNTs with metal nanoparticles, continuous plating of the SWCNTs was achieved with an increased concentration of the positive valency metal species solution. It is believed that with a lower feed concentration of the positive valency metal species the activated sites on the surface of the SWCNT that are non-uniform along the axis of the nanotubes promote nanoparticle decoration, while at higher concentrations of the positive valency metal species, growth initially begins on these same activated sites and follows the axial direction of the SWCNT resulting in a continuous plating of metal.

It is also possible to prepare dendritic nanoparticles with the method of the present invention.

The dendritic nanoparticles are prepared by subjecting nanotubes, a coating precursor, and one or more reagents capable of reacting with the coating precursor and preparing the coating, to shear, thereby causing production of a dendritic coating on the nanotubes from said reaction under shear. The concentration of the coating precursor-containing solution must be sufficient to promote growth in respective lateral and axial directions from activation sites on the nanotubes. A high concentraton of the coating precursor results in a randomly ramified hyper-branched coating on the nanotube core.

Dendritic metal-coated nanoparticles can be prepared by preparing a dendritic metal coating on a core comprising a nanotube.

Transition from one dimensional quantum confinement to zero-dimension quantum dot behaviour for SWCNTs is predicted by cutting CNTs into short lengths. CNTs can be semi-metallic, semi-conducting or metallic, depending on the wrapping angle and tube diameter/dimension. Prior art methods for cutting SWCNTs are extremely tedious or use high temperature treatments: with a scanning tunnelling microscope and cutting SWCNTs by partially fluorination and pyrolyzing the partially fluorinated nanotubes in an inert atmosphere or vacuum close to 1000° C.

The method of the present invention for preparing metal coated nanotubes is also usefully applied to cut the metal coated nanotubes into short lengths. The process for cutting nanotubes comprises preparing coated nanotubes and subjecting said coated nanotubes to shear. The process for cutting nanotubes of the present invention is particularly effective in regard to metal coated nanotubes.

The inventors have noted that TEM analysis of coated nanotubes prepared according to the present invention under shear revealed shortening/cutting of the nanotubes into smaller lengths when compared to CNTs decorated with nanoparticles. CNTs twisted as a result of shearing and centrifugal forces in the region of shear. While CNTs have been reported to produce reversible elastic distortions in the presence of strong radial forces, the twisting and buckling of the CNTs becomes highly amplified in regions of shear. During the process of coating the CNTs, the regions of the CNTS that are coated show low or no radial distortion, while the uncoated regions produce amplified radial elastic response. Severe buckling and twisting around these sites results in lateral fractures in the CNTs. Additionally, the possible Stone-Wales transformation under shear may lead to bond rotation defects. The dynamics of these defects are dictated by the tube chirality as well as the applied tension and temperature. In particular at high applied strain and low temperature all tubes are extremely brittle and may lead to lateral fracture.

Nanoparticles comprising a core of a first metal and a coating of a second metal can be prepared by subjecting nanoparticles of the first metal, a second metal precursor and one or more reagents capable of reacting with the second metal precursor and preparing the second metal, to shear.

Further, nanoparticles comprising a core of a metal and a shell or coating of a metal compound can be prepared by subjecting nanoparticles of the metal, a metal compound precursor, and one or more reagents capable of reacting with the metal compound precursor and preparing the metal compound, to shear.

Notably, the metal core of the aforementioned metal-metal compound nanoparticles can be removed therefrom by treatment with a leaching agent, such as an inorganic acid (e.g. HCl, HNO₃, H₂SO₄, HClO₄), thereby resulting in the production of a nanoparticle comprising a shell of the metal compound.

Nanoparticles of a first metal compound and a second metal compound can be subsequently prepared by subjecting the nanoparticles comprising the shell of the first metal compound with a second metal compound precursor, and one or more reagents capable of reacting with the second metal compound precursor and preparing the second metal compound, to shear, thereby causing production of nanoparticles of the first and second metal compounds from said reaction under shear. In the reaction under shear, the second metal compound is deposited in a void disposed within the shell of the first metal compound, thereby forming a core of the second metal compound coated with the shell of the first metal compound.

According to the methods of the present invention, the coating precursor is a compound which is converted into the coating when the compound is reacted with one or more reagents capable of converting the compound into the coating.

The reaction between the coating precursor and the one or more reagents includes, but is not limited to, an electrochemical or redox reaction, an acid-base reaction, a metal-ligand complexation reaction, a host-ligand complexation reaction, a synthetic reaction, a decomposition reaction, an exchange reaction, a precipitation reaction, photochemical reaction, microwave-induced reaction, and so forth. The person skilled in the art can formulate suitable reactions for production of metals, alloys, and inorganic compounds.

Further, said reaction includes, but is not limited to, liquid-liquid, solute-liquid, liquid-solid, solute-solid, liquid-gas and solute-gas reactions.

In one embodiment of the invention, the product of the coating precursor conversion reaction is a metal or alloy. In another embodiment of the invention, the product of the reaction is an inorganic compound.

In some embodiments of the invention where the coating is a metal, the precursor comprises a positive valency metal species. Suitable examples of positive valency metal species in respect of the noble and platinum metals include, but are not limited to, Ag(I), Au(I), Au(III), Pd(II), Pd(IV), Pt(II), Pt(IV), Rh(III), Ru(II), Ru(III) and Os(II). In some embodiments the positive valency metal species are stabilised in solution as metal-ligand complexes or metal compounds.

In one embodiment, the one or more reagents capable of reacting with the positive valency metal species into the metal coating material comprise a reductant, in particular a reductant having a redox potential suitable for reducing the positive valency metal species. The reductant can be in the solid, liquid, or gaseous state. In one particular embodiment the reductant is gaseous hydrogen. In another particular embodiment the reductant is ascorbic acid, glucose or reducing carbohydrate-like molecules. In an alternative embodiment the reductant is a metal hydride.

The delivery of the therapeutic compositions can be controlled (pulsed or sustained) by coating nano-particles, which is possible using the present invention, as is the ability to control the formation of nano-structures of higher complexity. Coating nano-particles with materials can slow down the delivery, and the particles can be coated with more drug. Moreover, by mixing different nanoparticles with and without coatings it may be possible to regulate release of the therapeutic compositions in a controlled way, thereby optimising the pharmacokinetics.

Nano-particles can be more versatile in developing sustained or pulsed drug delivery, limiting fluctuations in plasma drug concentration levels, reducing adverse reactions and dosing frequency leading to improved patient compliance and therapeutic outcomes, and reduced particle size allows drugs to be more efficiently delivered to the deep lung.

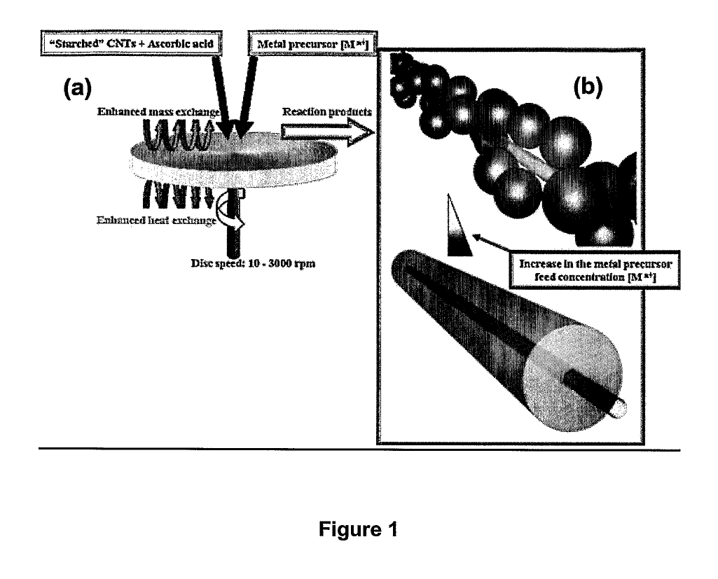

The coated nanoparticles described in the present specification have been produced according to the methods of the present invention using a rotating surface reactor as shown in FIG. 1.

Non-limiting examples of rotating surface reactors suitable for use in the processes of the present invention are disclosed in PCT/GB00/00519, PCT/GB00/00523, PCT/GB00/00524, PCT/GB00/00526, and PCT/GB01/00634, the full disclosures of which are hereby incorporated into the present application by reference. Rotating surface reactors may be in the form of spinning disc reactors, spinning cone reactors, rotating tube reactors, and other shaped reactors as discussed in the above patent applications. Non-limiting examples of methods of operation of said rotating surface reactors suitable for use in the processes of the present invention are disclosed in PCT/GB02/03368 the full disclosure of which is also hereby incorporated into the present application by reference.

The key components of a rotating surface reactor includes: (I) a rotating surface with controllable speed, and (ii) feed jets located at a predetermined radial distance from the centre of the surface. A thin fluid film (1 to 200 pm) is generated on a rapidly rotating surface (10 to 3000 rpm), within which coating of the nanoparticle occurs (FIG. 1). The nanoparticles, the coating precursor-containing solution and the one or more reagents for reacting with the coating precursor are delivered to the rotating surface where they are accelerated by viscous drag until an inverse hydrostatic jump occurs and the thin fluid film spreads across the rotating surface. In a preferred embodiment, the nanoparticles are conveniently provided as a liquid dispersion thereof.

In forming nano-particles the resulting super-saturations and large surface areas at the interface of mixing lead to high nucleation rates and rapid particle growth, affording mono-dispersed nano-particles with very narrow size distributions, amongst other properties. For spinning disc reactors, the residence time on the disc is typically 0.5 second, which is predetermined by the rotation speed of the disc and viscosity of the solutions.

Rotating tube reactors are a variance of spinning disc reactors with the ability to control the residence time of reactions on the rotating surface. A thin film of liquid will reside in the tube with the thickness governed by its viscosity, the height of a ridge at the end of the tube, and the rotational speed. Adding more liquid to one end will slowly force some liquid out the opposite end, and thus by controlling the rate of addition it is now possible to control the residence time.

Both spinning disc reactors and rotating tube reactors have many control parameters, including disc speed, feed rates, concentrations, temperature, introducing otherwise immiscible gases, surface texture of the rotating surface to enhance mixing, and more.

Moreover, highly exothermic reactions are more easily controlled on rotating surface which allows high heat transfer, and the amount of solvent used can therefore be limited, thereby minimising the amount of waste the technology generates.

Preferably, the rotation speed of the rotating surface is at least 10 rpm. More preferably, the rotation speed of the rotating surface is between 10 rpm and 3000 rpm. More preferably still, the rotation speed of the rotating surface is between 300 rpm and 3000 rpm. More preferably still, the speed of the rotating surface is between 1000 rpm and 3000 rpm. More preferably still, the rotation speed of the rotating surface is about 2500 rpm. It will be appreciated that the preferred speed will be influenced by the composition of the nanoparticle core compound and its surface, the features of the rotating surface, and the processes used to coat the nanoparticles with another compound.

It will be appreciated that the speed of rotation of the rotating surface is related to the degree of shear and that higher speeds will create higher shear. It will be further appreciated that the preferred rotation speed may depend on the type of rotating surface reactor employed.

The strong shearing forces and viscous drag between the moving fluid layer and the rotating surface give rise to highly efficient turbulent mixing within the fluid layer. The intensity of the mixing pays a role in the precipitation mechanism and consequently in the coating thickness and uniformity. The fluid on the surface of the disc has a short and controllable residence time and results in a rapid induction time for nucleation and growth of nanoparticles.

In addition, the thinness of the fluid film contributes to many influential chemical processing characteristics, one being a very high surface area to volume ratio, resulting in more favourable interactions between the film and its surroundings. Thin layering permits uniform heat transfer throughout the entire reaction mixture whereas the ability for such heat conduction and convection is absent in a batch reactor.

The thinness of the film contributes to many influential chemical processing characteristics, one being a very high surface area to volume ratio, resulting in more favourable interactions between the film and its surroundings. The thin film permits uniform heat transfer throughout the entire reaction mixture whereas the ability for such efficient heat conduction and convection is absent in a batch reactor. In addition, strong shearing forces create turbulence and break the surface tension of the film, making waves and ripples. These waves and ripples add to the vigour of mixing and combining the nanoparticles, the coating precursor-containing solution and the one or more reagents for reacting with the coating precursor, enabling very high heat and mass transfer rates in the film. This in turn ensures extremely short reaction residence times enabling impulse heating and immediate subsequent cooling, and plug flow identifying even mixing and transfer through the entire film.

The residence time of reagents on the rotating surface and the speed of rotation of the rotating surface can be varied along with the feed rate and concentrations of both the liquid dispersion of nanoparticles and the coating precursor, and any other solution of reagents for reacting with the coating precursor. For example, depending upon the time necessary for the reaction to reach completion, the viscosity of the reaction mixture, or the rotational velocity can be adjusted to shorten or lengthen the residence time of the nanoparticles, the coating precursor-containing solution and one or more reagents for reacting with the coating precursor on the rotating surface or the thickness of the thin fluid film formed on the rotating surface. In one embodiment, the rotating surface has a velocity in the range of 10-3000 rpm.

Advantageously, a feature of rotating surface process technology is that it is convenient for continuous flow production which facilitates scale up, avoiding the use of batch technology where there is often the need to separate mixtures.

Rotating surface processing (FIG. 1) can employ a rapidly rotating (10-3000 rpm) disc manufactured from 316 stainless steel or other material with PTFE composite seals onto which surface reagents can be delivered through a number of different feed jets. Highly effective turbulent micro-mixing occurs as the reagents propagate across the rotating disc surface under the influence of centrifugal forces.

The reaction temperature is controlled by a recirculation coolant system that permits both heating and cooling of the rotating disc surface. Both smooth and grooved stainless steel rotating surfaces, or like surfaces constructed from other material, can be utilized for operation though grooved rotating surfaces have demonstrably superior wetting characteristics.

Effective throughputs range from 0.3 to 3.5 mL/s for low viscosity solvents delivered using continuous gear pumps and residence times within the reactor are commonly less than 1 second (in respect of 100 mm diameter rotating disc). Lower feeds and/or rotational speeds are prone to form rivulets rather than the requisite fluid film, whilst larger feed rates will require too large a spin up zone.

It will be appreciated that an advantage of the methods of the present invention is that the size, shape, phase and morphology of the coatings may be manipulated under continuous flow conditions.

### EXAMPLES

### Example 1

### Preparation of Metal Coated CNTs

The protocol for generating metal coated CNTs is illustrated in FIG. 1.

### Preparation of Solutions

A liquid dispersion of SWCNTs (as supplied by Cheap Tubes Inc.) (up to 3 g/L) was prepared by the addition of CNTs to aqueous starch-iodine solution (0.7 g/L) with excess L-ascorbic acid (0.3 M, Chem. Supply). The excess L-ascorbic acid reduces excess iodine in the liquid dispersion of CNTs, and acts as the reagent which reacts with the coating precursor and forms the coating.

The aqueous iodine-starch solution (0.7 g/L) was prepared by adding starch (0.07 g; Ajax Chemicals) in water (10 mL; >18 MS/cm, Millipure Milli-Q system) to boiling water (90 mL) and continuously stirring until a colourless solution was obtained. A KI/I₂ solution (standard Lugol's iodine solution) was added drop-wise to the starch solution until a typical dark blue colouration was retained.

In the example, the coating precursors were silver, gold, and platinum positive valency species. Aqueous solutions (>18 Macm, Millipure Milli-Q system) of silver, gold, and platinum positive valency species were prepared from AgNO₃, HAuCl₄, and HPtCl₆ (AGR Matthey), respectively, at various concentrations in the range 0.5-15 mM.

### Preparation of Coated Nanotubes

Integrated feed pumps were used to feed (0.5 mL/s) of the liquid dispersion of the SWCNTs as described above and the coating precursor solutions onto a rotating surface (2500 rpm) of a spinning disc reactor (100 series; Protensive, Inc). The product was collected from the reactor for analysis. The size and morphology of the resultant coated nanotubes were examined by transmission electron microscopy (TEM JEOL 3000F and JEOL 2000FX II).

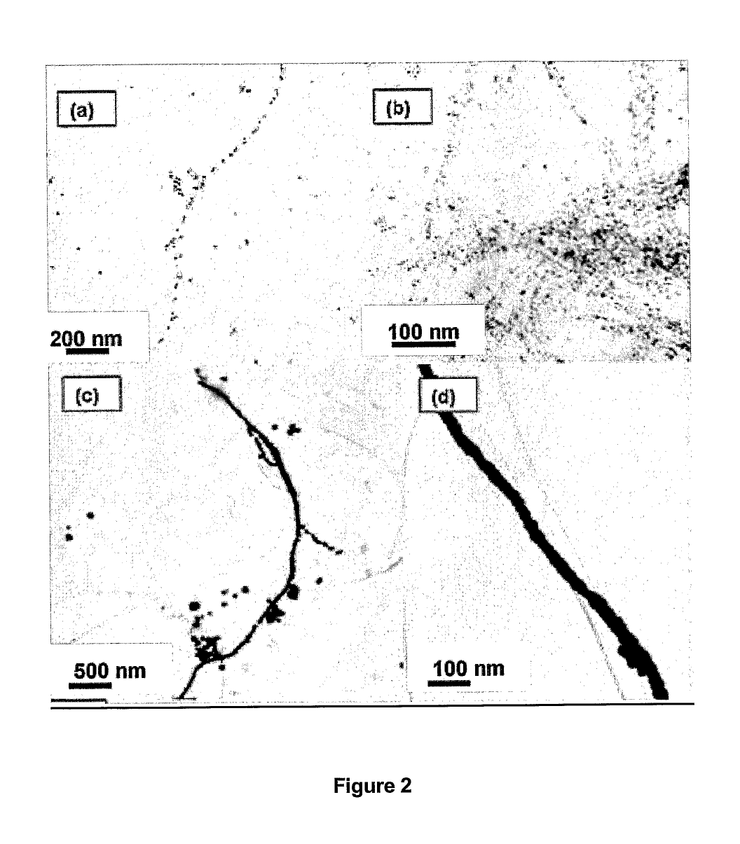

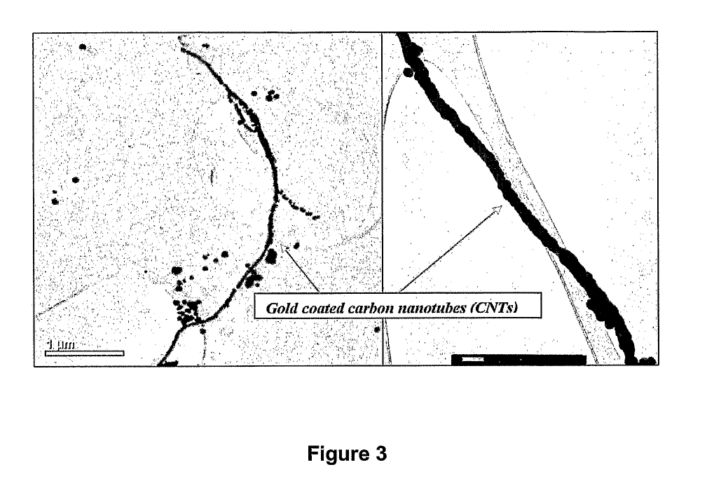

FIGS. 2 (a)-(d), 3, 4, and 5 demonstrate that nanotubes were coated with Au, Pt, and Ag. Whilst lower concentrations of the coating precursor solutions resulted in decoration of SWCNT with metal nanoparticles, higher concentrations provided continuous metal coatings on the SWCNT.

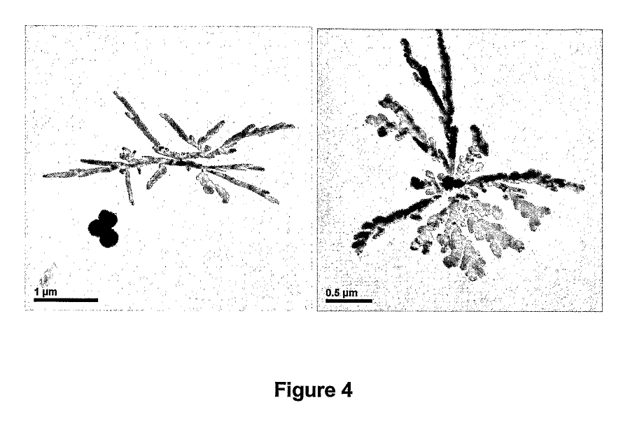

In FIG. 4, the observed dendritic nanoparticles, specifically a dendritic silver coating bound to a SWCNT, arose with increased concentration of the coating precursor solution (15 mM).

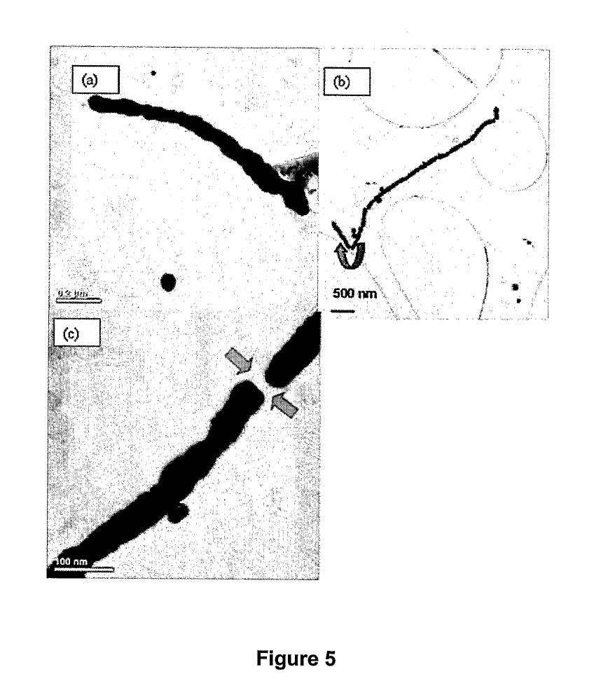

FIG. 5 demonstrates that metal coated nanoparticles can be laterally fractured or cut in regions of high shear. FIGS. 5(a) and 5(b) are TEM micrographs of a fractured gold coated SWCNT prepared from a HAuCl₄ solution (10 mM), while FIG. 5(c) is a TEM micrograph of uncoated sites along the SWCNT that propagate fracture under shear.

### Example 2

### Preparation of Silver Coated One Dimensional Nanoparticles of Fullerene C₆₀

A liquid dispersion of one dimensional nanoparticles of fullerene C₆₀ were prepared by addition of unground fullerene C₆₀ (approximately 10 mg; 95%, BuckyUSA) to aliquots of aqueous starch-iodine solution (75 mL; 0.7 g/L) and the reaction mixtures stirred overnight. The iodine-starch solutions were prepared as described. L-Ascorbic acid (0.3 M, Chem. Supply) was subsequently added to the reaction mixture in excess.

The resulting liquid dispersion of fullerene C₆₀, and aqueous solution of silver nitrate (15 mM), were simultaneously fed (0.5 mL/s) to a rotating surface (2500 rpm) of the rotating surface reactor described above via first and second integrated feed pumps, respectively. The product was collected from the reactor for analysis. The size and the morphology of the resultant coated nanotubes were examined by transmission electron microscopy (TEM JEOL 3000F and JEOL 2000FX II).

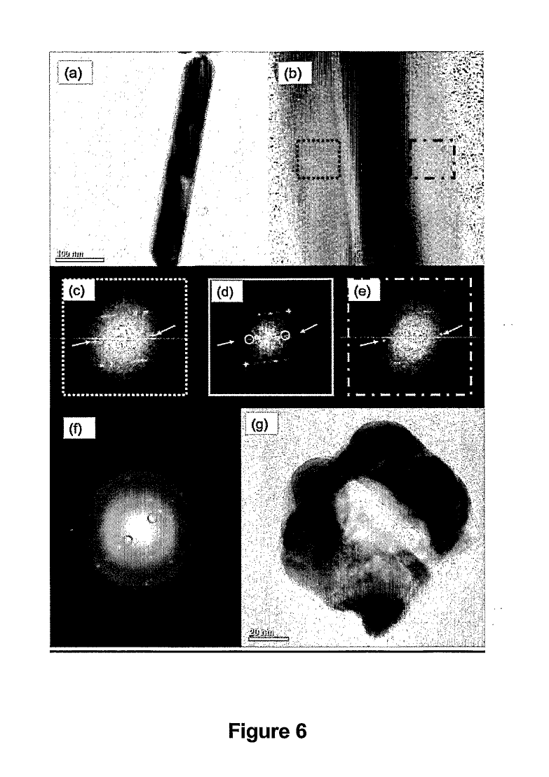

The one dimensional nanoparticles of fullerene C₆₀ were coated with silver by intensively mixing a liquid dispersion of the nanoparticles with silver nitrate solution under conditions of high shear. Excess ascorbic acid was used to reduce the iodine in the starch-iodine complex and to convert Ag(I) to Ag(O). Starch is used as a very effective anti-agglomeration agent for preparation of the one dimensional nanoparticles of fullerene C₆₀, enabling adequate wettability to ensure complete and uniform covering by the silver coating (see FIG. 6(a)). The diameter of the silver coating can be varied by regulating the initial concentration of silver nitrate, at the same time maintaining the integrity of fullerene C₆₀ nanoparticles under conditions of high shear.

High resolution TEM shows an intense interference pattern along the centre of the coated nanoparticle (FIG. 6(b)). This has been attributed to the interference arising from the fullerene C₆₀ core and the polycrystalline nature of the silver cross-section. The corresponding FFT patterns of the silver coated fullerene C₆₀ nanoparticle along the lateral direction are represented in FIGS. 6(c)-(e). Analysis of the FFT pattern showed the typical 4H spacing for silver along the edges (FIGS. 6(c) and 6(e)) and the centre (FIG. 6(d)), as indicated by the arrows. However, the FFT pattern along the centre (FIG. 6(d)) showed the appearance of the atypical 0.39 nm spacing (as indicated by the circles) corresponding to the fullerene C₆₀ reflections observed in uncoated fullerene C₆₀ nanoparticles. Indeed, the presence of the principal reflections at 0.39 nm in the electron diffraction pattern (FIG. 6(f)) confirmed the presence of the fullerene C₆₀ core. While the appearance of the higher order spots in the FFT pattern along the centre (FIG. 6(d)) that do not appear on the electron diffraction pattern (FIG. 6(g)) is believed to be a Moire interference fringe effect arising from the overlap between the fullerene C₆₀ core and the silver coating spacings.

Analysis of the silver reflections (FIG. 6(f)) confirmed hexagonal structural packing. The presence of the hexagonal structure when compared to the traditional fcc packed bulk structure has been attributed to the size dependent phase transformation in silver. The size effect arises from the fact that 4H silver nano-wires have a more favourable surface configuration but higher volume internal energy than fcc silver nano-wires. The cross section of the silver nano-wires was observed by embedding them in resin and microtoming through the wires (FIG. 6(g)). The cross sectional image confirms the polycrystalline nature of the nano-wires, and the hexagonal structure of the individual subcrystals.

### Example 3

### Preparation of Gold Coated Silver Nanoparticles

A liquid dispersion of silver nanoparticles was prepared by reacting an aqueous solution of silver nitrate (15 mM) with excess L-ascorbic acid in a 0.03 wt % starch solution under conditions of high shear. Integrated feed pumps were used to feed (0.5 mL/s) of the silver nitrate solution and the ascorbic acid and starch solution onto a rotating surface (2500 rpm) of a spinning disc reactor 100 series (Protensive, Inc).

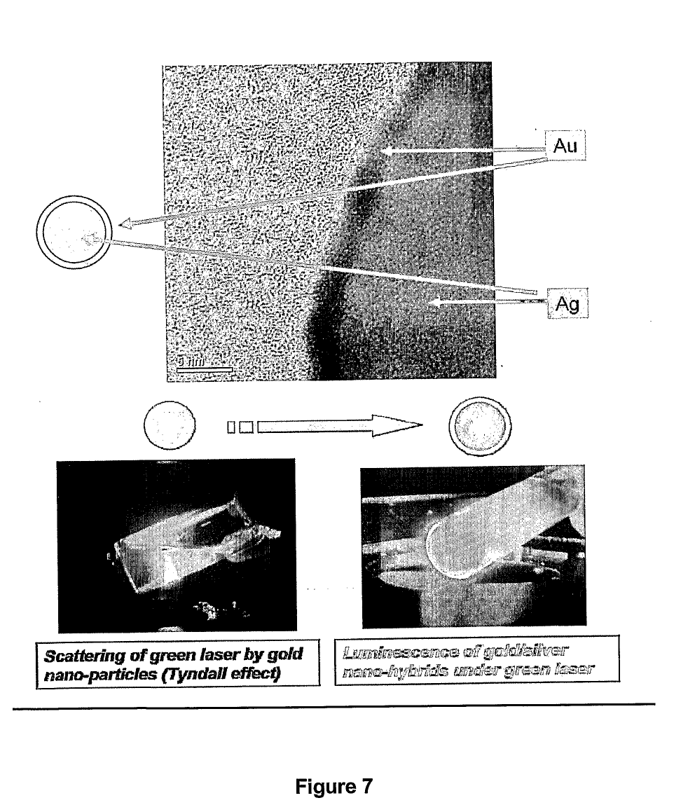

The silver nanoparticles formed were centrifuged and redispersed in a HAuCl₄ solution (15 mM). The liquid dispersion of silver nanoparticles in the HAuCl₄ solution and an aqueous solution of L-ascorbic acid, were simultaneously fed (0.5 mL/s) to the rotating surface (2500 rpm) of a rotating surface reactor described above via first and second integrated feed pumps, respectively. The resultant nanoparticles comprise a silver core with a gold coating, as shown in FIG. 7.

### Example 4

### Preparation of Titania Coated Silver Nanoparticles

A liquid dispersion of silver nanoparticles was prepared by reacting an aqueous solution of silver nitrate (15 mM) with excess L-ascorbic acid in a 0.03 wt % starch solution under conditions of high shear. Integrated feed pumps were used to feed (0.5 mL/s) of the silver nitrate solution and the ascorbic acid and starch solution onto a rotating surface (2500 rpm) of a spinning disc reactor 100 series (Protensive, Inc).

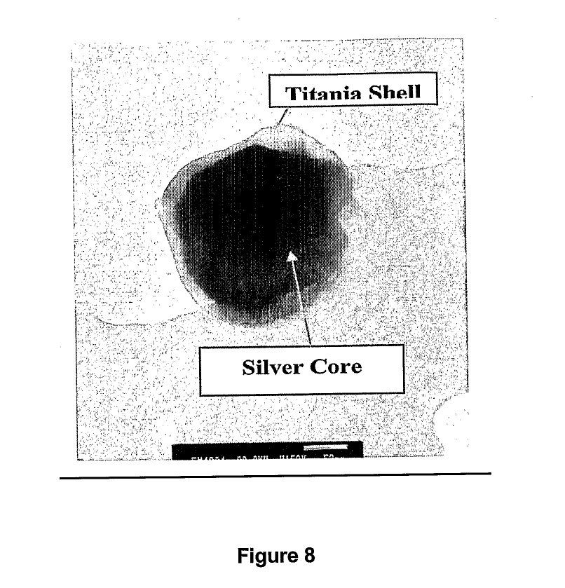

The silver nanoparticles formed were centrifuged and redispersed in water. The aqueous dispersion of silver nanoparticles and a solution of titanium tetraisopropoxide in dry ethanol (0.03 wt %) were simultaneously fed (0.5 mL/s) to the rotating surface (2500 rpm) of a rotating surface reactor described above via first and second integrated feed pumps, respectively. The resultant nanoparticles comprise a silver core with a titania coating, as shown in FIG. 8.

### Example 5

### Preparation of Nanoparticles of Titania and Mixed Titania/Iron Oxide

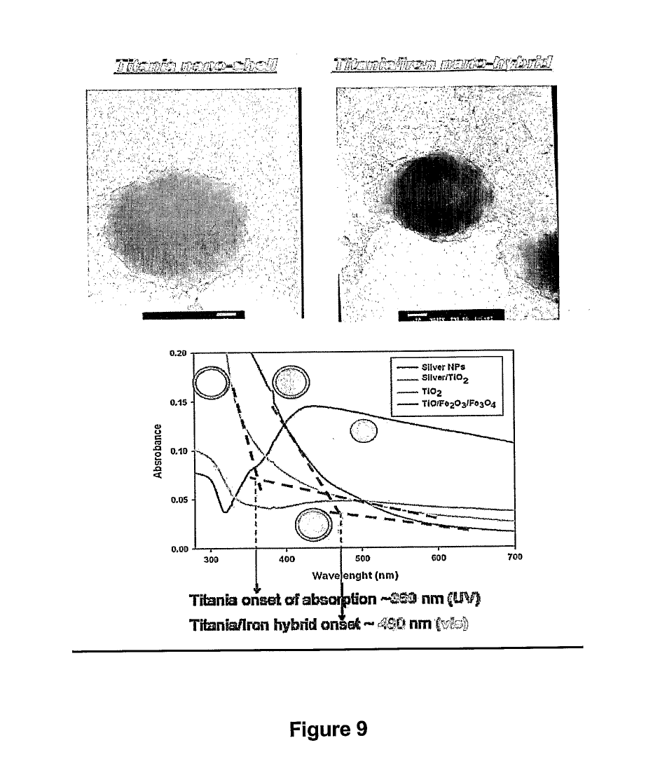

Nanoparticles of titania were prepared by treating the titania coated silver nanoparticles from Example 4 with hydrochloric acid (2 M) to dissolve the silver core, leaving a hollow TiO₂ shell, as shown in FIG. 9.

The nanoparticles comprising a hollow TiO₂ shell were purified and redispersed in dilute hydrochloric acid. The liquid dispersion of titania nanoparticles and a ferric chloride solution (10 mM) were simultaneously fed (0.5 mL/s) to the rotating surface (2500 rpm) of a rotating surface reactor described above via first and second integrated feed pumps, respectively. The resulting nanoparticles comprised a core of Fe₂O₃/Fe₃O₄ with a titania coating, as shown in FIG. 9.

The silver nanoparticles formed were centrifuged and redispersed in water. The aqueous dispersion of silver nanoparticles and a solution of titanium tetraisopropoxide in dry ethanol (0.03 wt %) were simultaneously fed (0.5 mL/s) to the rotating surface (2500 rpm) of a rotating surface reactor described above via first and second integrated feed pumps, respectively. The resultant nanoparticles comprise a silver core with a titania coating, as shown in FIG. 8.

### Example 6

### Preparation of Iron Coated Cnts

SWCNTs (10 mg) were dispersed in a 1:1 mixture of 70% HNO₃ and 98% H₂SO₄ (5 mL) in a reaction chamber in a CEM Focused Microwave Synthesis System (Discover Model). The microwave power was set at 300 W, pressure 12 bar and temperature 130° C. for 30 min. After the reaction, the SWCNTs were filtered, washed and re-dispersed in ultrapure Milli-Q water (100 mL) and sonicated for 15 min.

The functionalised SWCNTs were dispersed in water and the container purged with N₂ gas to remove oxygen. FeCl₂.4H₂O (10 mM) and FeCl₃.6H₂O (20 mM) (1:2 molar ratios) were added and the mixture stirred for 1 hr. The solution was filtered to remove excess Fe^{2+/3+} and the resulting carbon nanotube and Fe^{2+/3+} complex re-dispersed in deoxygenated Milli-Q water.

Integrated feed pumps were used to feed a suspension of CNTs and Fe^{2+/3+} (0.5 mL/s) from one feed and deoxygenated NH₄OH from another feed under an atmosphere of high purity argon gas to a spinning disc reactor 100 series (Protensive, Inc).

Ultrafine (2-3 nm) of Fe3O4 nanoparticles with very narrow size distribution were observed by TEM to be uniformly coated onto the CNTs surface

1. A method of coating nanoparticles comprising subjecting nanoparticles, a coating precursor and one or more reagents to shear, wherein the coating precursor and the one or more reagents react to provide a coating on the nanoparticles.

2. The method of claim 1, wherein the nanoparticles are nanotubes.

3. The method of claim 1, wherein the coated nanoparticles are substantially continuously coated.

4. The method of claim 1, wherein the nanoparticles, the coating precursor and the one or more reagents are subjected to shear on the rotating surface of a rotating surface reactor.

5. (canceled)

6. The method of claim 4, wherein the coating precursor and one or more reagents are directed separately to the rotating surface of the rotating surface reactor.

7. The method of claim 4, wherein the coating precursor and one or more reagents are combined with the nanoparticles prior to directing the coating precursor and one or more reagents to the rotating surface of the rotating surface reactor.

8. The method of claim 4, wherein the nanoparticles are provided to the rotating surface reactor as a liquid dispersion thereof.

9. The method of claim 4, wherein the rotating surface reactor spins at a speed sufficient to cause the combined liquid dispersion of nanoparticles, coating precursor, and the one or more reagents to spread over the rotating surface as a continuously flowing thin film.

10-14. (canceled)

15. The method of claim 1, wherein the nanoparticle is a metal, an alloy, a metalloid, a metal compound such as a metal oxide, an inorganic compound, a carbon-based material or a therapeutic composition.

16. The method of claim 15, wherein the metal is selected from the group consisting of noble or platinum metals transition metals and main group metals.

17. The method of claim 15, wherein the alloy is selected from the group consisting of alloys of noble metal and transition metals, and noble metal alloys.

18. The method of claim 15, wherein the inorganic compound is selected from the group consisting of SiO₂ and metal compounds.

19. The method of claim 15, wherein the carbon-based material is selected from the group consisting of carbon nanotubes, one-dimensional nanoparticles of fullerene C₆₀, and three-dimensional nanoparticles of fullerene C₇₀.

20. The method of claim 15, wherein the therapeutic composition is selected from the group consisting of biologies, amino acids, proteins, peptides, nucleotides, nucleic acids, and analogs thereof.

21-23. (canceled)

24. The method of claim 2, wherein the nanotubes include carbon nanotubes, inorganic nanotubes or peptidyl nanotubes.

25. (canceled)

26. The method of claim 1, wherein the coating is a metal, an alloy, a metalloid, a metal compound, an inorganic compound, or a carbon-based material.

27. The method of claim 26, wherein the metal is selected from the group consisting of noble metals, transition metals and main group metals.

28. The method of claim 26, where the coating is a metal and wherein the precursor comprises a positive valency metal species selected from the group consisting of Ag(I), Au(I), Au(III), Pd(II), Pd(IV), Pt(II), Pt(IV), Rh(III), Ir(III), Ru(II), Ru(III) and Os(II).

29-31. (canceled)

32. The method of claim 26, wherein the alloy is selected from the group consisting of alloys of noble metal and transition metals, and noble metal alloys.

33. The method of claim 26, wherein the inorganic compound is selected from the group comprising SiO₂, and metal compounds.

34-35. (canceled)

36. The method of claim 26, wherein the nanoparticle is a first therapeutic composition and the coating is a second therapeutic composition.

37-39. (canceled)

40. The method of claim 1, wherein the method further comprises the step of adding an anti-agglomeration agent.

41-47. (canceled)

48. The method of claim 2, wherein the method comprises the further step of cutting nanotubes into shortened lengths.

49. The method of claim 1, wherein the method further comprises the step of substantially removing the nanoparticle core of the coated nanoparticle to provide a nanoparticle shell.

50. The method of claim 49, wherein the method further comprises the step of subjecting nanoparticle shells, a coating precursor and one or more reagents to shear, wherein the coating precursor and the one or more reagents react to provide a coating on the nanoparticle shells.

51-52. (canceled)

53. A method of preparing dendritic coated nanoparticles comprising subjecting nanotubes, a coating precursor, the concentration of the coating precursor being sufficient to promote growth in respective lateral and axial directions from activation sites on the nanotubes and one or more reagents to shear, wherein the coating precursor and the one or more reagents react to provide a dendritic coating on the nanotubes.

54-55. (canceled)

56. A pharmaceutical composition comprising nanoparticles coated in accordance with the method of claim 1.
